(19) United States
(12) Patent Application Publication
(10) Pub. No.: US 2003/0134267 A1
(43) Pub. Date: Jul. 17, 2003

(54) Sensor for detecting biomolecule using carbon nanotubes

(76) Inventors: Seong-Ho Kang, Gyeonggi-do (KR); Yukeun Eugene Pak, Gyeonggi-do (KR); Won-Bong Choi, Gyeonggi-do (KR)

Correspondence Address: CANTOR COLBURN, LLP, 55 GRIFFIN ROAD SOUTH, BLOOMFIELD, CT, 06002

(21) Appl. No.: 10/240,227

(22) PCT Filed: Aug. 13, 2002
(86) PCT No.: PCT/KR02/01544

(30) Foreign Application Priority Data
Aug. 14, 2001 (KR) 2001-49033

(51) Int. Cl. C12Q 1/00; C12Q 1/68; C12M 1/34

(52) U.S. Cl. 435/4; 435/6; 435/287.2

(57) ABSTRACT

Provided is a sensor for detecting a biomolecule, and particularly, a sensor for detecting a biomolecule, including (a) a substrate; and (b) a plurality of carbon nanotubes which are arranged on the substrate and provide a binding site for a receptor for a target biomolecule. With the biomolecular sensor, a various kinds of disease-associated biomolecules can be detected simultaneously, accurately and quickly.

### BACKGROUND OF THE INVENTION

1. Field of the Invention

The present invention relates to a bio-chip, and more particularly, to a high-throughput, nanoarray-type bio-chip which is highly integrated in nanoscale.

2. The Description of the Related Art

As a result of the human genome project, the human genomic sequence was reported on February 2001 (“The Sequence of the Human Genome”, J. Craig Venteret al., Science, 291, 1304-1351, 2001). However, genomics approaches have limitations in finding out an accurate mechanism of human disease processes and in treating human diseases. For this reason, proteomics approaches are gaining favor as an important area of research.

Most current approaches to diagnostic proteomics are concentrated on a microarray-based protein chip. Unlike the early-stage array technology using photolithography to form a polypeptide array on a substrate surface (Pirrung et al., U.S. Pat. No. 5,142,854, 1992, “Large scale photolithographic solid phase synthesis of polypeptides and receptor binding screening thereof”), a variety of approaches have been made to fabricate an array structure. In a variety of immunoassays, such as antigen-antibody pair assays and enzyme-linked immunosorbent assays, the need to develop a micro-array type format is gradually increasing. Comparing with DNA-based arrays, it is more difficult to minimize and highly integrate a protein-based array for higher sensitivity. A grid-like pattern for DNA oligonucleotides can be formed on a substrate surface by photolithography, but it is very difficult to form a grid pattern for an antibody which is a large protein having about 1,400 amino acids, to a high density for accurate diagnosis of diseases. Another limitation encountered with the manipulation of proteins is that their tertiary structure is susceptible to denaturation under denaturing conditions (Sandra Katzman, Anal. Chem., 14A-15A, 2001, “Chip-based mosaic immunoassays”; Andre Bernard, Bruno Michel, and Emmanuel Delamarche., Anal. Chem., 73, 8-12, 2001, “Microsaic Immunoassays”)

The key to these problems relies on the possibility of arranging a protein with a high resolution without denaturing its tertiary structure. A variety of approaches, including inkjet printing, drop-on-demand technique, microcontact printing, and soft lithography suggested by IBM, have been made to date. However, these techniques result in a spacing of from tens of micrometers to a few millimeters, and nanoarray-based protein chip highly integrated with natural proteins, i.g, without denaturing their tertiary structure, has not been developed yet.

Lieber et al. used carbon nanotubes, which are tubular, nano-sized carbon structures, in the manufacture of nano-sized microscopy probes (U.S. Pat. No. 6,159,742 (2002), Charles M. Lieber, Stanislaus S. Wong, Adam T Wooley, Ernesto Joselevich, “Nanometer-scale Microscopy Probes”). Eklund et al. produced stable iodine-doped carbon nanotubes or metallic nanoscale fibers (U.S. Pat. No. 6,139,919 (2000), “Metallic Nanoscale Fibers From Stable Iodine-doped Carbon Nanotubes”). Massey et al. synthesized electrochemiluminescent ruthenium complexes with functional group biomolecule-modified nanotubes (U.S. Pat. No. 5,866,434 (1999), Richard J. Massey et al., Graphitic Nanotubes in Luminescence Assays). However, applications of carbon nanotubes in the manufacture and development of a bio-chip have not been reported yet.

### SUMMARY OF THE INVENTION

To overcome the above problems, it is an object of the present invention to provide a bio-chip integrated with carbon nanotubes in nanoarray-type.

It is another object of the present invention to provide a high-throughput assay method for different kinds of biomolecules using the bio-chip.

The term of “sensor for detecting a biomolecule” throughout this specification and claims is intended to mean a “bio-chip” in terms of its structure including a plurality of receptors bound on a one substrate.

According to an aspect of the present invention, there is provided a nanoarray-type sensor for detecting a biomolecule comprising: (a) a substrate; and (b) a plurality of carbon nanotubes which are arranged on the substrate and provide binding sites for a receptor for a target biomolecule.

In a nanoarray-type bio-chip according to the present invention for diagnostic purpose, carbon nanotubes are arranged on a substrate, and an electric field of an opposite polarity to a net charge of the receptors is applied to some or all of the carbon nanotubes to selectively move receptors for diagnostic target biomolecules to a desired carbon nanotbues and to bind them there to a desired position at a high-density.

In another aspect, the present invention provides a multi-channel-type sensor for detecting a biomolecule comprising: (a) a substrate; (b) micro- or nano-sized multiple channels disposed in the substrate; and (c) one or more carbon nanotubes arranged at a particular position in the multiple channels and io provide the binding sites for a receptor for a biomolecule.

In a multi-channel-type sensor for detecting a biomolecule according to the present invention, one or more carbon nanotubes are disposed at a desired position in each of the multiple channels, and an electric field of an opposite polarity to a net charge of each receptor is applied to each of the carbon nanotubes. Analogous to the nanoarray-type sensor for detecting a biomolecule according to the present invention, different kinds of receptors can be selectively attached to the carbon nanotubes within each of the multiple channels.

In a multi-channel-type sensor for detecting a biomolecule according to the present invention, multiple channels can be formed directly on a silicon substrate by photolithography etching or can be formed by attaching a separate glass or other substrate on which multiple channels have been formed, to a surface of a silicon substrate.

According to the present invention, suitable materials for the substrate include a variety of polymeric substances, such as silicon, glass, molten silica, plastics, and polydimethylsiloxane (PDMS), and carbon nanotubes of several to hundreds of nanometers are arranged on the substrate in a nanoarray.

According to the present invention, the receptors are biological substances capable of acting as probes that are detectable when bound to the target biomolecules. Suitable receptors include nucleic acids, proteins, peptides, amino acids, ligands, enzyme substrates, cofactors, and oligosaccharides.

According to the present invention, a target biomolecule, which binds to a receptor, is a biomolecule of interest to be analyzed. The target biomolecule may be proteins, nucleic acids, enzymes, or other boimolecules capable of binding to the receptor. More preferably, the target biomolecule is a disease-associated protein.

According to the present invention, a carbon nanotube array on the substrate can be fabricated using a well-known, conventional carbon nanotube synthesis technique. For example, after forming a plurality of cavities of a diameter of a few nanometers on a dielectric layer, for example, of alumina, at an interval of a few nanometers, carbon nanotubes are vertically grown through the cavities by a chemical vapor deposition method, an electrophoretic method, or a mechanical method.

According to the present invention, each of the carbon nanotubes is connected through at least one conductive nanowire to a power source from which an electrical charge is applied. Here, the conductive nanowire can be formed of a single molecule (Leo Kouwenhoven, “Single-Molecule Transistors”, Science Vol., 275, pp. 1896-1897, Mar. 28, 1997, which is incorporated herein by reference). The conductive nanowire may be deposited in the chip fabrication process prior to growing the carbon nanotubes.

According to the present invention, one or more kinds of receptors are selectively immobilized on the individual carbon nanotubes by applying an electric field having polarity opposite to a net charge of each receptor at constant or different levels to the carbon nanotubes.

Alternatively, one receptor may be immobilized on two or more carbon nanotubes if necessary. In this case, an electrical charge of the same polarity or an opposite polarity can be applied to the carbon nanotubes on which one kind of receptor is immobilized,

According to the present invention, immediately before or after binding of the receptors to the carbon nanotubes, an auxiliary binder may be treated to enhance a binding force of the carbon nanotubes and the receptors. This auxiliary binder maintains the binding of the carbon nanotubes and the receptors after the electrical field applied to the carbon nanotubes is removed.

According to the present invention, suitable auxiliary binders include a chemical having a functional group, such as aldehyde, amino, or imino at its carbonyl end, a monolayer of, for example, SiO₂ or Si₃N₄, a membrane of, for example, nitrocellulose, and a polymer, for example, polyacrylamide gel or PDMS.

A bio-chip according to the present invention may further include a detection system for detecting the binding of the receptors on the carbon nanotubes or the binding of the target biomolecules to the receptors. The detection system may be included in or separated from the bio-chip.

A bio-chip according to the present invention may utilize a well-known internal detection system, for example, an electrical detector, a resonance detector, or a detector using a saw sensor or a cantilever. Preferably, the internal detection system may use an electrical detection method. In the method of detecting an electrical signal, binding of the receptors or biomolecules to the carbon nanotubes is detected by reading a minor change in voltage level of the carbon nanotubes occurring when the receptors or biomolecules are bound to the carbon nanotubes, using an appropriate circuit.

When a bio-chip according to the present invention utilizes an external detection system, an optical detection method, such as a fluorescence detection method including an x-y fluorescent laser detection method or laser-desorption-ionic mass spectroscopy, a laser-induced fluorescence detection method, an absorption detection method, a resonance detection method, and an interference detection method, can be applied. In the x-y fluorescent laser detection method, the samples bound to the receptors are reacted with fluorescent molecules or fluorescence-labeled antibodies, and thus reacted entire chip is placed on an x-y fluorescence laser detector to detect fluorescence.

A multi-channel-type sensor for detecting biomolecules according to the present invention may further include a delivery and separation system in each of the multiple channels to deliver and separate the biomolecules according to their size and electrical properties.

According to the present invention, the delivery and separation system may use a micro fluid flow control method well known in the field by using, for example, a micro-pump or capillary electrophoresis device.

According to another aspect of the present invention, there is provided a high-throughput assay method for analyzing various kinds of disease-associated biomolecules using only one sensor for detecting a biomolecule described above. The method directly detects various kinds of disease-associated target proteins bound to various kinds of receptors or measurs a difference in binding force of the target proteins to the receptors.

When the multi-channel-type sensor for detecting biomolecules according to the present invention described above is used, target proteins bound to specific receptors immobilized on the multiple channels can be directly detected, or the mobility or retention time of target molecules is measured from the difference in their interaction with the receptors, so that various kinds of diseases can be simultaneously diagnosed on a mass scale using only one chip. In this case, since the binding force of the biomolecules and receptors varies depending on the electrochemical properties of the biomolecules, mobility is an important factor to qualify and quantify the biomolecules.

According to the present invention, protein-specific receptors, which are specific to disease-associated target proteins, can be selectively immobilized on the carbon nanotubes arranged in a nanoarray on a chip with the application of an electric field. Various kinds of receptors capable of interacting with various kinds of disease-associated target proteins can be selectively immobilized by applying electric fields having different polarity to the individual carbon nanotubes. As a result, it is possible to simultaneously, accurately, and quickly diagnose various kinds of diseases using only one chip.

Alternatively, according to the present invention, after attaching carbon nanotubes to multiple channels, one or more receptors are immobilized on the carbon nanotubes at a desired position in each of the multiple channels. Different channels may have different receptors. Next, target proteins bound to the receptors are directly detected, or a difference in a mobility of target proteins due to their interactions with the receptors is measured. As a result, various kinds of diseases can be easily, accurately, and quickly diagnosed using only one chip including multiple channels.

### BRIEF DESCRIPTION OF THE DRAWINGS

The above objects and advantages of the present invention will become more apparent by describing in detail preferred embodiments thereof with reference to the attached drawings in which:

FIG. 1 illustrates principles of forming vertical carbon nanotubes;

FIG. 2 is a photograph of carbon nanotubes in different shapes;

FIG. 3 is a perspective view of a nanoarray-type sensor for detecting biomolecules according to the present invention;

FIG. 4 is a top view of a multi-channel-type sensor for detecting biomolecules according to the present invention;

FIG. 5 illustrates interactions between target proteins and various kinds of receptor probes in a nanoarray-type sensor for detecting biomolecules according to the present invention; and

FIG. 6 illustrates interactions between target proteins and various kinds of receptor probes in a multi-channel-type sensor for detecting biomolecules according to the present invention.

### DETAILED DESCRIPTION OF THE INVENTION

Hereinafter, the present invention will be described in greater detail with reference to the attached drawings.

Embodiment 1: Synthesis of Carbon Nanotubes

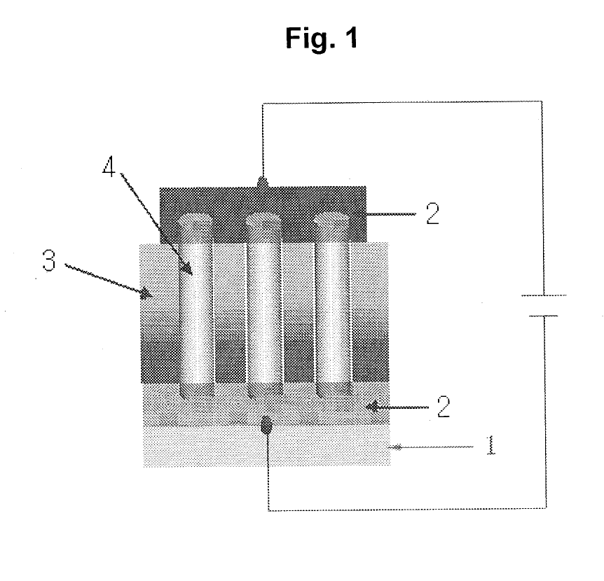

FIG. 1 illustrates principles of vertically growing carbon nanotubes on a substrate coated with a conductive layer. As shown in FIG. 1, a conductive layer 2 is formed on a substrate 1 and a dielectric layer 3, for example, formed of alumina, is formed on the conductive layer 2. After forming a plurality of cavities of a few nanometers through a dielectric layer 3 at an interval of a few nanometers, the carbon nanotubes 4 are vertically grown through the cavities by a chemical vapor deposition method, an electrophoretic method, or a mechanical method.

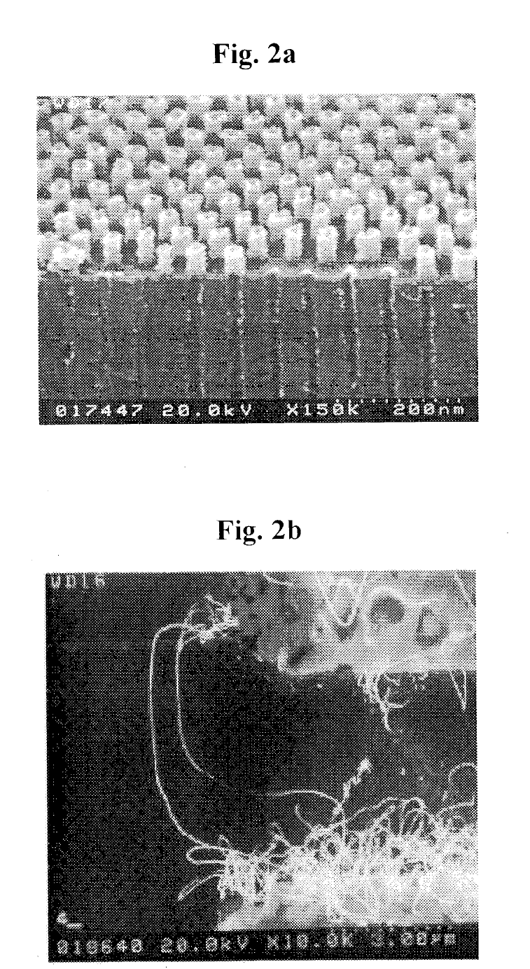

FIG. 2 is a photograph of carbon nanotubes in different shapes. As is apparent from FIG. 2, carbon nanotubes have different shapes depending on their fabrication method. Vertically grown carbon nanotubes are shown in FIG. 2A, and horizontally grown carbon nanotubes are shown in FIG. 2B. It is preferable to vertically grow carbon nanotubes of a nanoscale diameter on a non-conductive substrate using a carbon nanotube-based vertical transistor fabrication method. A plurality of cavities of a diameter of several to hundreds of nanometers are formed in a dielectric layer, for example, formed of alumina, at an interval of several to hundreds of nanometers, and carbon nanotubes are vertically aligned through the nano-sized cavities by a chemical vapor deposition method, an electrophoretic method, or a mechanical method. The vertical carbon nanotubes are used as channels. Using a semiconductor fabrication method, a gate electrode is formed around each of the carbon nanotubes, with source and drain electrodes atop and below each of the carbon nanotubes. As a result, nano-sized vertical carbon nanotube transitors that can be switched electrically are formed.

Embodiment 2: Nanoarray-Type Bio-Chip

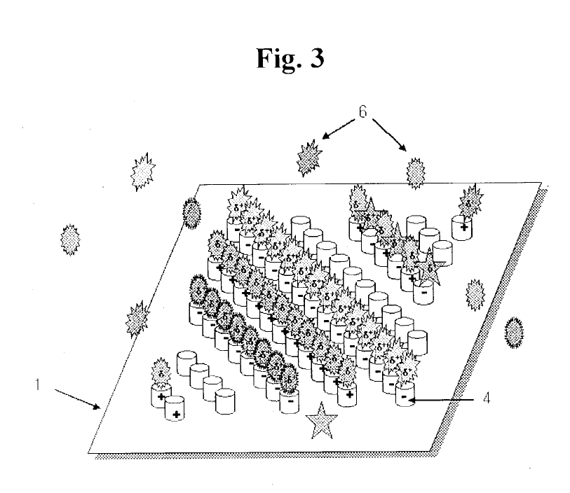

FIG. 3 is a perspective view of a nanoarray-type bio-chip according to the present invention, in which carbon nanotubes are nano-arrayed on a substrate, and various kinds of receptors are selectively immobilized on the carbon nanotubes at a particular position on the chip. As shown in FIG. 3, unlike conventional methods using lithography and spotting techniques, electric fields having different polarity are applied to the carbon nanotubes 4 arranged on a substrate 1 in nanoscale intervals to selectively move or immobilize the receptors 6 having a net charge opposite to the applied electric field, on the carbon nanotubes 4. The substrate 1 for the chip may be formed of a variety of materials.

In particular, each of the carbon nanotubes 4 formed in Embodiment 1 is utilized as one electrode. An electrical charge of a polarity opposite to the net charge of different kinds of receptors 6, such as proteins, peptides, amino acids, and other biological molecules, is selectively applied to the carbon nanotubes 4 to move or immobilize particular receptors 6 on the carbon nanotubes 4 at a particular position. The receptors 6 are bound to carbon nanotubes using an auxiliary binder, such as a variety of chemicals, monolayers, or polymers. As a result, a bio-chip in a nanoarray (10⁻⁹), which has a higher density than a conventional microarray (10⁻⁶) structure, is obtained.

The receptors 6, such as proteins, peptides, and amino acids, have a specific isoelectric point (pI) and a neutral, positive, or negative net charge depending on the ionic concentration or pH of the solution (Seong Ho Kang, Xiaoyi Gong, Edward S. Yeung, Anal. Chem., (2000), 72(14), 3014-3021, “High-throughput Comprehensive Peptide Mapping of Proteins by Multiplexed Capillary Electrophoresis”; Landers, J. P. Handbook of Capillary Electrophoresis, CRC Press Boca Raton, Fla., 1997; pp. 219-221). The conditions of the receptor solution are changed to control electrostatic interaction or hydrophobic interaction between the receptors 6 and charged carbon nanotubes 4 to thereby selectively move or immobilize one or more kinds of receptors 6 on the carbon nanotubes 4 at a particular position on the chip.

Embodiment 3: Multi-Channel-Type Bio-Chip

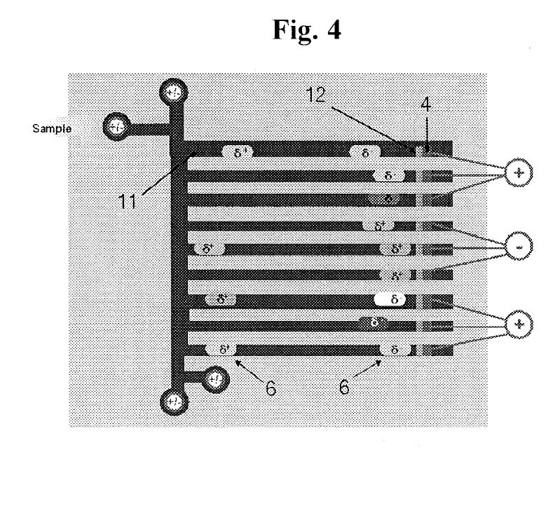

FIG. 4 is a top view of a multi-channel-type bio-chip according to the lo present invention, in which multiple channels are formed in the chip, carbon nanotubes are arrayed at a particular position in the channels, and various kinds of receptors are selectively immobilized on the carbon nanotubes at a particular position on the chip. As shown in FIG. 4, unlike conventional methods using lithography and spotting techniques, an electric field is applied to carbon is nanotubes 4 arranged in nanoscale intervals in the multiple channels 11 formed in a substrate 1 to selectively move or immobilize receptors 6 having a net charge opposite to the applied electric field, on the carbon nanotubes 4 at a particular position on the chip. The substrate 1 for the chip may be formed of a variety of materials.

In particular, after forming the multiple channels 11 of a micro- or nano-size in the substrate 1, one or more carbon nanotubes 4 are arrayed at a desired position in each of the channels 11. Next, an electric field is applied to the carbon nanotubes 4 to selectively immobilize different kinds of receptors 6 for each of the channels 11. A sample is injected through one end of the channels 11, a hydrodynamic flow is induced using a micro-pump to deliver the sample into the channels 11. Alternatively, an electric field may be applied to both ends of the channels 11 to deliver the sample by capillary electrophoresis. A variety of diseases can be identified simultaneously, accurately, and quickly by directly detecting a target biomolecule in the flow, bound to the particular receptors 6 attached to a particular position within the channels 11, or by measuring the mobility or retention time of the target molecules from the difference in their interaction with the receptors 6. The above-described structure of the multi-channel-type bio-chip of the present invention can be applied in manufacturing a variety of bio-chips, including a comprehensive high-throughput protein-chip capable of assaying a living biological sample in a liquid state, including protein, while maintaining the activity of the biological sample, by selectively moving or immobilizing specific receptors 6 on the carbon nanotubes at a particular position within the channels 11.

Embodiment 4: Detection System

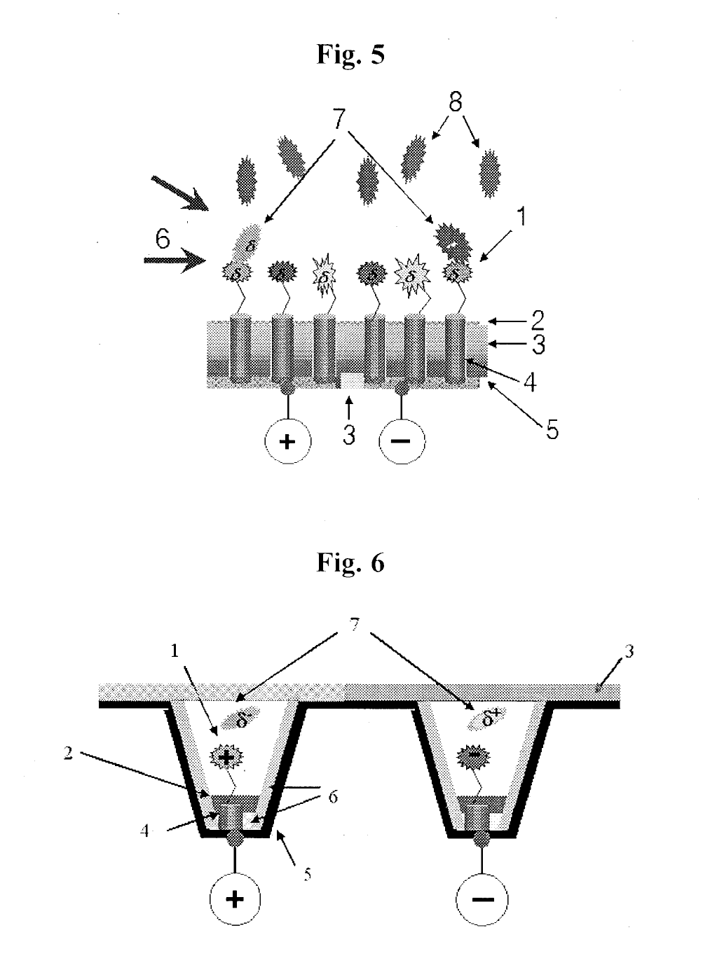

FIG. 5 illustrates interactions between diagnostic target proteins and various kinds of receptor probes immobilized on the carbon nanotubes arrayed in nanoscale intervals at a high-density. FIG. 6 illustrates interaction between target proteins and different kinds of receptor probes immobilized on the carbon nanotubes arrayed within multiple channels. As shown in FIG. 5, after dropping a sample solution containing diagnostic target proteins 7 onto the chip to which various kinds of receptor probes 6 have been attached, the target proteins 7 bound to the receptor probes 6 are directly detected, or the interaction between the target proteins 7 and the receptor probes 6 immobilized on the carbon nanotubes is measured, so that different kinds of diseases can be diagnosed simultaneously. Referring to FIG. 6, a sample solution containing target proteins 7 is delivered into a desired position within the multiple channels by using a micro-pump or by capillary electrophoresis, to which receptor probes 6, which are different for each of the multiple channels, have been attached. Next, the target proteins 7 bound to the receptor probes 6 are directly detected, or the mobility or retention time of the target proteins 7 due to their interaction with the receptor probes 6 is measured, so that different kinds of diseases can be diagnosed simultaneously. Bovine serum albumin 5 protects the target proteins 7 from interacting with materials other than the receptor probes 6, such as the substrate.

In the present invention, a detection system for detecting the binding of receptors and carbon nanotubes or the binding of receptors and biomolecules may be further included. These types of binding can be detected by an electrical method or resonance method or by using an x-y fluorescent laser reader. When the method of detecting an electrical signal is applied, the binding of receptors or biomolecules is detected by reading a minor change in voltage level of the carbon nanotubes occurring when the receptors or biomolecules are bound to the carbon nanotubes, using an appropriate circuit. When the resonance detection method is applied, a nanoplate structure designed to have a resonance frequency of a range from megaHertzs to low gigaHertzs is irradiated with a laser diode, and the binding of receptors or biomolecules to the nanoplate structure is optically measured by detecting a reflection signal using a position detection photodiode. When the x-y fluorescent laser reader is used, the target biomolecules bound to receptors are reacted with, for example, fluorescent molecules or fluorescence-labeled antibodies, and the entire chip after the reaction with the target biomolecules is placed on the x-y fluorescent laser reader to detect fluorescence. In particular, the entire chip is scanned with a laser beam capable of exciting the fluorescence-labeled target proteins and imaged by using a charge-coupled device (CCD) capable of scanning the entire chip array. Alternatively, a confocal microscope, which increases automation and detects data rapidly at a high resolution, can be applied to collect data from the chip array.

In a multi-channel-type bio-chip according to the present invention, a sample including proteins is flowed into each of the multiple channels 11 while one or more carbon nanotubes 4 are attached to each of the multiple channels 11. An electrical signal from each of the carbon nanotubes 4 and parameters, such as protein separation rate (depending on the size and charge of the proteins) and the duration of retention of the proteins on the carbon nanotubes (hereinafter, “retention time”, depending on the electrical properties of the proteins), are measured by using a microcontroller or microprocessor for controlling the flow rate within each of the channels 11. The smaller the protein molecular size is, the higher the separation rate is. A higher degree of matching between the proteins and receptors extends the retention time. Therefore, the separation time (an initial point of time at which a protein is detected after injection of the sample) and the retention time are crucial parameters for the identification of the protein. Prior to injecting a sample to be assayed into the detection system, a known protein can be injected into the detection system as a reference for calibration purpose. The two parameters are protein-specific parameters. A signal-specific profile of each standard protein may be stored in a memory to be compared with that of the tested sample.

As described above, according to the present invention, a nanoarray-based protein-chip can be manufactured using carbon nanotubes at a higher density compared with conventional microarray-based protein-chips. Since a very high-density nanoarray is mounted on a single chip, many kinds of the human proteins and their variants can be simultaneously assayed using only one protein-chip according to the present invention.

According to the present invention, each of the carbon nanotubes can be used as one electrode. Therefore, specific receptors can be selectively moved or immobilized on the carbon nanotubes at a particular position with the application of a constant level or different levels of an electric field to the carbon nanotubes. In other words, various kinds of receptors can be attached to one chip at a high density, so that different kinds of diseases can be simultaneously identified. It is possible to develop a comprehensive high-throughput bio-chip by attaching a different receptor for each of the carbon nanotubes arranged in nanoscale intervals on a single chip.

In a multi-channel-type bio-chip according to the present invention, a specific-receptor protein is migrated to and adsorbed-at a desired position within the multiple channels by electrophoresis. Accordingly, various kinds of receptors can be easily immobilized on the carbon nanotubes within each of the channels without denaturing their tertiary structure. Naturally occurring biological receptors can be loaded and integrated into the single bio-chip at a high density without denaturing their tertiary structure. In addition, a binding position of the receptors can be adjusted so that the active site of the receptors is exposed.

According to the present invention, it is possible to develop a variety of quality nanoarray-based bio-chips, such as DNA-chips, PCR-chips, or protein-chips.

In addition, since a bio-chip according to the present invention is based on the electrical interaction between the carbon nanotubes and the receptors, the bio-chip can be reused by inverting the charge of the carbon nanotubes to unbind the carbon nanotubes and receptors and washing the bio-chip with a solution after completion of a test. Alternatively, the carbon nanotubes and receptors may be unbound from one another by heating the entire bio-chip to induce protein denaturation.

What is claimed is:

1. A sensor for detecting a biomolecule comprising:
(a) a substrate; and
(b) a plurality of carbon nanotubes which are arranged on the substrate, and provide a binding site for a receptor for a target biomolecule.

2. The sensor for detecting a biomolecule of claim 1, wherein the substrate is formed of a material selected from the group consisting of silicon, glass, molten silica, plastics, and polydimethylsiloxane (PDMS).

3. The sensor for detecting a biomolecule of claim 1, wherein the receptors are selected from the group consisting of nucleic acids, proteins, peptides, amino acids, ligands, enzyme substrates, cofactors, and oligosaccharides.

4. The sensor for detecting a biomolecule of claim 1, wherein the target biomolecule is selected from the group consisting of proteins, nucleic acids and enzymes.

5. The sensor for detecting a biomolecule of claim 1, further comprising a detection system for detecting the immobilization of the receptors on the carbon nanotubes or the binding of the biomolecules to the receptors.

6. The sensor for detecting a biomolecule of claim 5, wherein the detection system comprises an electrical detector or an x-y fluorescent laser reader.

7. A sensor for detecting a biomolecule comprising:
(a) a substrate;
(b) micro- or nano-sized multiple channels formed in the substrate; and
(c) one or more carbon nanotubes which are arranged at a particular position within the multiple channels, and provide a binding site for a receptor for a target biomolecule.

8. The sensor for detecting a biomolecule of claim 7, wherein the substrate is formed of a material selected from the group consisting of silicon, glass, molten silica, plastics, and polydimethylsiloxane (PDMS).

9. The sensor for detecting a biomolecules of claim 7, wherein the receptors are selected from the group consisting of nucleic acids, proteins, peptides, amino acids, ligands, enzyme substrates, cofactors, and oligosaccharides.

10. The sensor for detecting a biomolecule of 7, wherein a target biomolecule is selected from the group consisting of proteins, nucleic acids and enzymes.

11. The sensor for detecting a biomolecule of claim 7, further comprising a detection system for detecting the immobilization of the receptors on the carbon nanotubes or the binding of the biomolecules to the receptors.

12. The sensor for detecting a biomolecule of claim 11, the detection system comprises an electrical detector or an x-y fluorescent laser reader.

13. The sensor for detecting a biomolecule of claim 7, wherein each of the multiple channels further comprises a delivery and separation system to deliver and separate the biomolecules to be assayed according to their size and electrical properties.

14. The sensor for detecting a biomolecule of claim 13, wherein the delivery and separation system comprises a micro-pump or a capillary electrophoretic device.

15. A method for manufacturing a sensor for detecting a biomolecule comprising: forming a plurality of cavities of a diameter of several to hundreds of nanometers at an interval of several to hundreds of nanometers in a dielectric layer; and vertically growing a plurality of carbon nanotubes through the plurality of cavities.

16. A method for detecting a biomolecule comprising: forming a plurality of carbon nanotubes on a substrate; applying a constant or different levels of an electric field of a polarity opposite to a net charge of each of receptors, to the carbon nanotubes in order to move and bind a receptor that specifically interacts with a target biomolecule to a corresponding carbon nanotubes; applying a sample to the receptor bound carbon nanotubes; and detecting the target biomolecule bound to the receptor to determine the target biomolecule.

17. The method of claim 16, further comprising, before or after immobilizing the receptors on the corresponding carbon nanotube, treating an auxiliary binder to enhance an adhesion between the carbon nanotubes and the receptors.

18. The method of claim 17, wherein the auxiliary binder is selected from the group consisting of a chemical having an aldehyde, amino, or imido group at its carbonyl end, a SiO₂ monolayer, a Si₃N₄ monolayer, a nitrocellulose membrane, polyacrylamide gel and polydimethylsiloxane (PDMS).

19. The method of claim 16, wherein different kinds of target biomolecules are simultaneously analyzed on a mass scale by directly detecting the target biomolecules bound to various kinds of receptors or by measuring a difference in binding force of the target molecules to the receptors.

20. The method of claim 16, wherein the target biomolecule is a disease-associated target protein.
